(19) United States
(12) Patent Application Publication
(10) Pub. No.: US 2020/0305473 A1
(43) Pub. Date: Oct. 1, 2020

(54) Method and Composition for Reducing Pathogens in Rendered Food Products Using Lactic Acid Bacteria

(71) Applicant: Texas Tech University System, Lubbock, TX (US)

(72) Inventors: Mindy M. Brashears, Wolfforth, TX (US); David Campos, Muleshoe, TX (US); Kendra Nightingale, Lubbock, TX (US); Guy Loneragan, Wolfforth, TX (US); Markus F. Miller, Abernathy, TX (US)

(21) Appl. No.: 16/310,981

(22) PCT Filed: Jun. 28, 2017
(86) PCT No.: PCT/US17/39669

§ 371 Date: Dec. 18, 2018

Related U.S. Application Data
(60) Provisional application No. 62/355,416, filed on Jun. 28, 2016.

(51) Int. Cl. A23L 3/3571; A23L 33/135; A23L 33/19; A01N 63/22; C12R 1/225; C12N 1/20

(52) U.S. Cl. CPC A23L 3/3571; A23L 33/135; A23L 33/19; A23V 2002/00; C12R 1/225; C12N 1/20; A23Y 2220/79; A01N 63/22

(57) ABSTRACT

The present invention provides a method for inhibiting the growth of food-borne pathogens, nosocomial pathogens and spoilage microorganisms, the method comprising the steps of: contacting at least one lactic acid bacterium strain selected from the group consisting of Lactobacillus salivarius (L14, L28 and FS56), a mixture thereof; or a whey obtained from fermentation of the lactic acid bacterium strain with the microorganisms.

### TECHNICAL FIELD OF THE INVENTION

The present invention relates in general to the field of reducing pathogens in food products, specifically to compositions of matter and methods of making and using lactic acid bacterium that produces protease-sensitive antimicrobial agents at low storage temperatures for bioprotection of refrigerated products.

### BACKGROUND OF THE INVENTION

Without limiting the scope of the invention, its background is described in connection with reducing transmission of food borne illness. Bacterial contamination of food products is known to be responsible for spoilage and for the transmission of food borne illness. This problem is particularly important in processed and ready to eat meats and dairy products which are not normally reheated by prior to ingestion and which are stored for extended times in refrigerators at 2-10° C. For example, Listeria monocytogenes is of particular concern in food products due to its tolerance of refrigeration temperatures, relatively high NaCl concentrations and anaerobic conditions or in products due to the lack of a heat inactivation step. As a result, a great deal of effort has been expended in attempts to identify natural products that can be safely added to foods for the purpose of inhibiting bacterial growth.

Antagonistic cultures added to food to inhibit pathogens and/or extend shelf life without changing the sensory properties of the product are termed “protective cultures”. In contrast to starter cultures, protective cultures are not intended to change the sensory properties of the product. Their use or that of their metabolic products (organic acids, hydrogen peroxide, enzymes and bacteriocins) is often referred to as “biopreservation” or “bioprotection”. Biopreservation is used as a method of preventing growth of spoilage bacteria and to ensure microbiological safety without changing the sensory characteristics of the product.

In addition, a re-growth of Listeria monocytogenes has often been observed with the use of bioprotective cultures after an initial phase of inhibition. Re-growth has been ascribed to the development of resistance of L. monocytogenes to the bacteriocins, degradation of bacteriocin molecules with endogenous proteases produced during the growth phase, adsorption of the bacteriocins to the surface of the producer strain, or specific interactions with the food matrix.

U.S. Patent Application Publication No. 2014/0213763, entitled “Lactic acid bacteria strain and its use for the protection of food products” discloses a strain of Lactobacillus curvatus as well as to compositions, cultures and food products thereof useful for preserving food products, especially under refrigerated conditions.

U.S. Patent Application Publication No. 7,744,868, entitled “Composition and method for inhibiting Salmonella and Campylobacter colonization in poultry” disclose the use of a novel competitive exclusion bacterial composition to prevent or reduce Salmonella or Campylobacter colonization in poultry.

### BRIEF SUMMARY OF THE INVENTION

The present invention provides a method for inhibiting the growth of food-borne pathogens, nosocomial pathogens and spoilage microorganisms, the method comprising the steps of: contacting at least one lactic acid bacterium strain selected from the group consisting of Lactobacillus salivarius (L14, L28 and FS56), a mixture thereof; or a whey obtained from fermentation of the lactic acid bacterium strain with the microorganisms. The microorganisms may be selected from the group consisting of gram-positive bacteria and gram-negative bacteria. The gram-positive bacteria are selected from the group consisting of Staphylococcus aureus, Listeria innocua, Listeria monocytogenes, Enterococcus faecium and Enterococcus faecalis. The gram-negative bacteria are selected from the group consisting of Escherichia coli and Salmonella Typhimurium. The Escherichia coli may be the O157:H7 serotype. The microorganisms may be selected from the group consisting of Aeromonas caviae; Aeromonas hydrophila; Aeromonas sobria; Bacillus cereus; Campylobacter jejuni; Citrobacter ssp.; Clostridium botulinum; Clostridium perfringens; Enterobacter ssp.; Enterococcus ssp.; Escherichia coli enteroinvasive strains; Escherichia coli enteropathogenic strains; Escherichia coli enterotoxigenic strains; Escherichia coli O157:H7; Klebsiella ssp.; Listeria monocytogenes; Plesiomonas shigelloides; Salmonella ssp.; Shigella ssp.: Staphylococcus aureus; Streptococcus ssp.; Vibrio cholerae; Yersinia enterocolitica. More preferably, the pathogenic-bacteria are Listeria monocytogenes. The at least one lactic acid bacterium strain may be Lactobacillus salivarius (L28), or strains MM13A, MM13B, P13, P17, Ecat, J19, J27, J35, J43, L14, L15, L17, L19, or LP28.

A method for reducing food-borne pathogens in a rendered food product comprising the steps of: combining a rendered food product and at least one lactic acid bacterium strain selected from the group consisting of Lactobacillus salivarius (L14, L28 and FS56), a mixture thereof; or a whey obtained from fermentation of the lactic acid bacterium strain with the microorganisms.

The microorganisms may be selected from the group consisting of Aeromonas caviae; Aeromonas hydrophila; Aeromonas sobria; Bacillus cereus; Campylobacter jejuni; Citrobacter ssp.; Clostridium botulinum; Clostridium perfringens; Enterobacter ssp.; Enterococcus ssp.; Escherichia coli enteroinvasive strains; Escherichia coli enteropathogenic strains; Escherichia coli enterotoxigenic strains; Escherichia coli O157:H7; Klebsiella ssp.; Listeria monocytogenes; Plesiomonas shigelloides; Salmonella ssp.; Shigella ssp.: Staphylococcus aureus; Streptococcus ssp.; Vibrio cholerae; Yersinia enterocolitica. More preferably, the pathogenic-bacteria are Listeria monocytogenes, or strains MM13A, MM13B, P13, P17, Ecat, J19, J27, J35, J43, L14, L15, L17, L19, or LP28. The rendered food product may be a meat, fatty meat trimmings, sausages, toothpaste, a grease, or soap.

The present invention provides a method for increasing the storage time of a food by reducing the spoilage microorganisms in contact with the food comprising the steps of: combining a food having one or more spoilage microorganisms with at least one lactic acid bacterium strain selected from the group consisting of Lactobacillus salivarius (L14, L28 and FS56), a mixture thereof; or a whey obtained from fermentation of the lactic acid bacterium strain with the one or more spoilage microorganisms to reduce the number of one or more spoilage microorganisms in contact with the food. The one or more spoilage microorganisms may be selected from the group consisting of Aeromonas caviae; Aeromonas hydrophila; Aeromonas sobria; Bacillus cereus; Campylobacter jejuni; Citrobacter ssp.; Clostridium botulinum; Clostridium perfringens; Enterobacter ssp.; Enterococcus ssp.; Escherichia coli enteroinvasive strains; Escherichia coli enteropathogenic strains; Escherichia coli enterotoxigenic strains; Escherichia coli O157:H7; Klebsiella ssp.; Listeria monocytogenes; Plesiomonas shigelloides; Salmonella ssp.; Shigella ssp.: Staphylococcus aureus; Streptococcus ssp.; Vibrio cholerae; Yersinia enterocolitica. More preferably, the pathogenic-bacteria are Listeria monocytogenes, or strains MM13A, MM13B, P13, P17, Ecat, J19, J27, J35, J43, L14, L15, L17, L19, or LP28.

The present invention provides a method for reducing a pathogenic load in rendered food products comprising the steps of: contacting at least one lactic acid bacterium strain selected from the group consisting of Lactobacillus salivarius (L14, L28 and FS56), a mixture thereof; or a whey obtained from fermentation of the lactic acid bacterium strain with the rendered food products having one or more pathogens to reduce the pathogenic load. The one or more pathogens may be selected from the group consisting of Staphylococcus aureus, Listeria innocua, Listeria monocytogenes, Enterococcus faecium Enterococcus faecalis, Escherichia coli and Salmonella Typhimurium. The microorganisms may be selected from the group consisting of Aeromonas caviae; Aeromonas hydrophila; Aeromonas sobria; Bacillus cereus; Campylobacter jejuni; Citrobacter ssp.; Clostridium botulinum; Clostridium perfringens; Enterobacter ssp.; Enterococcus ssp.; Escherichia coli enteroinvasive strains; Escherichia coli enteropathogenic strains; Escherichia coli enterotoxigenic strains; Escherichia coli O157:H7; Klebsiella ssp.; Listeria monocytogenes; Plesiomonas shigelloides; Salmonella ssp.; Shigella ssp.: Staphylococcus aureus; Streptococcus ssp.; Vibrio cholerae; Yersinia enterocolitica. More preferably, the pathogenic-bacteria are Listeria monocytogenes. The Escherichia coli may be the O157:H7 serotype. Alternatively, the bacterium is at least one of strains MM13A, MM13B, P13, P17, Ecat, J19, J27, J35, J43, L14, L15, L17, L19, or LP28

### BRIEF DESCRIPTION OF THE DRAWINGS

For a more complete understanding of the features and advantages of the present invention, reference is now made to the detailed description of the invention along with the accompanying figures and in which:

FIG. 1 shows the Lactic Acid Bacteriacolony morphology on MRS agar.

FIG. 2 is an image of the API Strip results L28 Fermentation.

FIG. 3 is an image of the API web software results.

FIG. 4 is an image of a spot agar test plate.

FIG. 5 shows a plot of the reduction of Salmonella on dry kibble pet food using novel lactic acid bacteria.

FIG. 6 is an image of the inhibition of Salmonella by lactic acid bacteria (L14, L28).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The term “bacteriocidal effect” as used herein refers to any type of treatment which effect the killing of bacteria (i.e. which reduce their numbers). This is in contrast to a “bacteriostatic effect” which refers to the situation where the treatment only inhibits the growth or reproduction of the bacteria. An agent is said to be a bactericide or a bacteriocide if the agent is able to kill one or more type of bacteria. A bacteriocide is said to possess bacteriocidal or bactericidal activity.

By “bacteriocins” we refer to peptides or protein molecules released extracellularly that are able to kill certain other closely related bacteria by a mechanism by which the producer cell exhibits a degree of specific immunity. The term “dairy product” is intended to include any food product made using milk or milk products, including, but not limited to, milk, yogurt, ice cream, cheese, butter, and cream.

As used herein, the expression “effective amount” refers to the amount of the invention which gives rise to an inhibition of the bacterial growth or a reduction of the number of other bacteria from the food product.

The term “food product” and “food stuff” as used herein refers to any food that is susceptible to spoilage as a result of bacterial growth and proliferation, e.g., but not limited to, meat, dairy products, vegetables, fruits and grains.

As used herein, the term “meat” refers to any meat product or meat by-product (including those processed) from an animal which is consumed by humans or animals, including, without limitation, meat from bovine, ovine, porcine, poultry, fish and crustaceous seafood. As used in the present application, the term “ready to eat meat product”, also referred to as RTE meat product, is intended to include any meat product which does not require cooking prior to consumption.

The terms “refrigerated product” or “preserved in a refrigerated state” are equally used and refer to food products which are stored at temperatures ranging from to 2 to 10° C. The food product can be packaged, packaged under vacuum or packaged at modified atmosphere.

As used herein the term “shelf life” means the period of time that a food product remains saleable to retail customers. In traditional meat processing, the shelf life of meat and meat by-products is about 30 to 40 days after an animal has been slaughtered. Refrigeration of meat during this period of time is expected to largely arrest and/or retard the growth of pathogenic bacteria, and to a lesser extent, spoilage bacteria. After about 30 to 40 days, however, refrigeration is no longer able to effectively control the proliferation of spoilage bacteria below acceptable levels.

The term “spoilage bacteria” as used herein refers to any type of bacteria that act to spoil food. Spoilage bacteria may grow and proliferate to such a degree that a food product is made unsuitable or undesirable for human or animal consumption. Bacteria are able to proliferate on food surfaces, such as meat surfaces, by assimilating sugars and proteins on such surfaces. By metabolizing these components, spoilage bacteria create by-products including carbon dioxide, methane, nitrogenous compounds, butyric acid, propionic acid, lactic acid, formic acid, sulfur compounds, and other undesired gases and acids. The production of such by-products alter the color of meat surfaces, often turning meat from a red color to a brown, grey or green color.

Gaseous by-products generated by spoilage bacteria also give spoiled meat an undesirable odor. The color and odor alterations of meat due to the growth of spoilage bacteria on a surface of a meat product often make such food product unsaleable to consumers.

In addition to the control of spoilage bacteria, another significant concern in the food processing industry is controlling the growth of food-borne pathogenic bacteria. As used herein, the term “food-borne pathogenic bacteria” refers to any food poisoning organism that is capable of causing disease or illness in animals or humans. The term “food-borne pathogenic bacteria” will be understood to include bacteria that infect the food product (for instance meat) and thereby cause disease or illness, as well as bacteria that produce toxins that cause disease or illness. Preferably, the food-borne pathogenic bacteria is selected from the group: Aeromonas caviae; Aeromonas hydrophila; Aeromonas sobria; Bacillus cereus; Campylobacter jejuni; Citrobacter ssp.; Clostridium botulinum; Clostridium perfringens; Enterobacter ssp.; Enterococcus ssp.; Escherichia coli enteroinvasive strains; Escherichia coli enteropathogenic strains; Escherichia coli enterotoxigenic strains; Escherichia coli O157:1-17; Klebsiella ssp.; Listeria monocytogenes; Plesiomonas shigelloides; Salmonella ssp.; Shigella ssp.: Staphylococcus aureus; Streptococcus ssp.; Vibrio cholerae; Yersinia enterocolitica. More preferably, the pathogenic-bacteria are Listeria monocytogenes.

Though significant improvements have been made in the past 20 years to reduce pathogenic load in foods intended for human consumption, concerns still exist regarding the health qualities of products such as animal feeds, soaps, toothpastes and the like that result from food rendering.

The disclosed technology proposes a method to reduce the pathogenic load of Salmonella and Listeria in rendered food products by introducing lactic acid bacteria. Specifically, a strain of Lactobacillus salivarius (L28) has been isolated and characterized that effectively reduces Salmonella and Listeria spp. in a variety of systems, including raw chicken fat and on stainless steel surfaces.

Identification of Lactic Acid Bacteria, Selection and evaluation of lactic acid bacteria as inhibitors of pathogenic bacteria. Screening environmental cattle fecal samples/Retail meat samples for lactic acid bacteria isolates that show antagonisms towards Salmonella, Escherichia coli and Listeria monocytogenes. The isolation of Lactic Acid Bacteria from samples was performed as follows. The samples were pretreated 10 g+90 ml Physiological water or BPW - - - stomacher, 2 min. A dilution series (10²-10⁸) was plated on MRS agar plates. The plates were incubated at 30° C. for 72 hrs. In all cases, the incubation period will need to be extended until visible colonies (white, small and round) appear. Colonies were randomly picked from plates containing 10-100 colonies with similar characteristics and were transferred to MRS broth and incubated 30° C. for 72 hrs. The fermentation MRS broth was streaked on MRS agar plates and incubated at 30° C. for 72 hrs under anaerobic condition to get final purified colonies. These isolates were sub-cultured twice (1% inoculum, 30° C., 24 hrs) in 10 ml MRS broth and kept frozen at −20° C. in MRS supplemented with 10% glycerol.

Screening for antimicrobial activity (Agar Well Diffusion Assay). Pathogenic and indicator microorganisms included: Salmonella (TSB broth, 30° C., 24 hrs), E. coli O157:H7 (TSB broth, 37° C., 18-24 hrs), Non-O157 STECs (TSB broth, 37° C., 18-24 hrs), and Listeria (BHI broth, 35° C., 18-24 hrs). The plates were evenly spread with each of indicator bacteria and drops (10 μl) of LAB cultures, growth in MRS broth (37° C., 18-24 hrs). Inhibition was recorded positive if a translucent halo zone was observed around the spot.

Confirming the positive Lactic Acid Bacteria strains. Cultured pathogenic bacteria included: Salmonella (TSB broth, 30° C., 24 hrs), E. coli O157:H7 (TSB broth, 37° C., 18-24 hrs), Non-O157 STECs (TSB broth, 37° C., 18-24 hrs), and Listeria (BHI broth, 35° C., 18-24 hrs). Pathogen and Lactic Acid Bacteria were inoculated into growth media. The samples were kept at refrigerated temperature and room temperature. Antimicrobial activity was analyzed and record under different temperatures.

Identification of Lactic Acid Bacteria. The positive Lactic Acid Bacteria strains were identified using the API 50 CH kit and analyzed by APILAB PLUS software (bioMerieux).

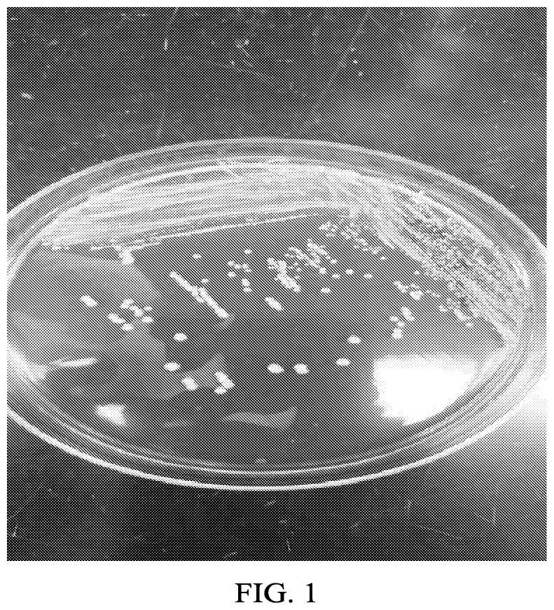

FIG. 1 shows the Lactic Acid Bacteria colony morphology on MRS agar: MRS agar results: Typical lactic acid bacteria colony produced by L28 is shown.

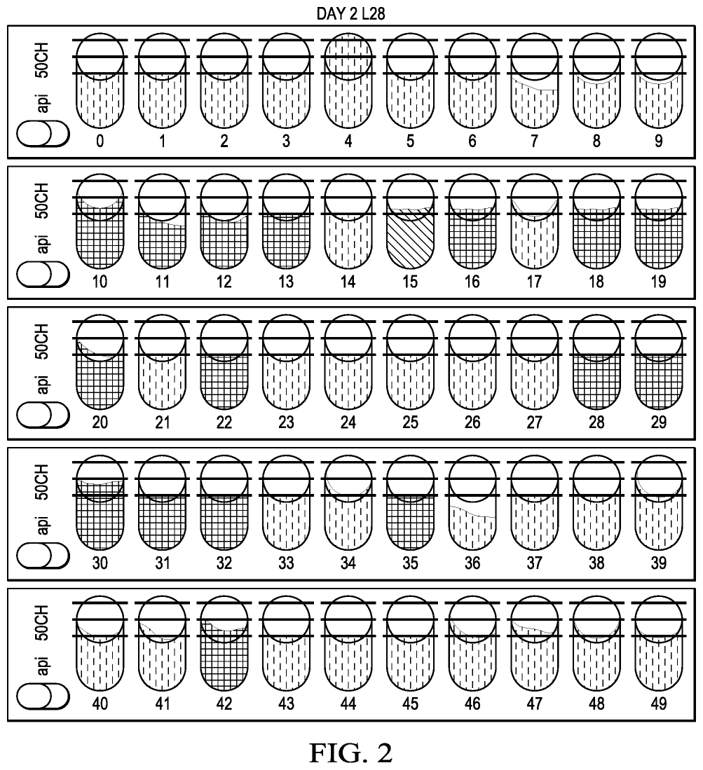

FIG. 2 is an image of the API Strip results L28 Fermentation of following substrates: Wells: 10, 11, 12, 13, 15, 16, 18, 19, 22, 28, 29, 30, 31, 32, 35, and 42.

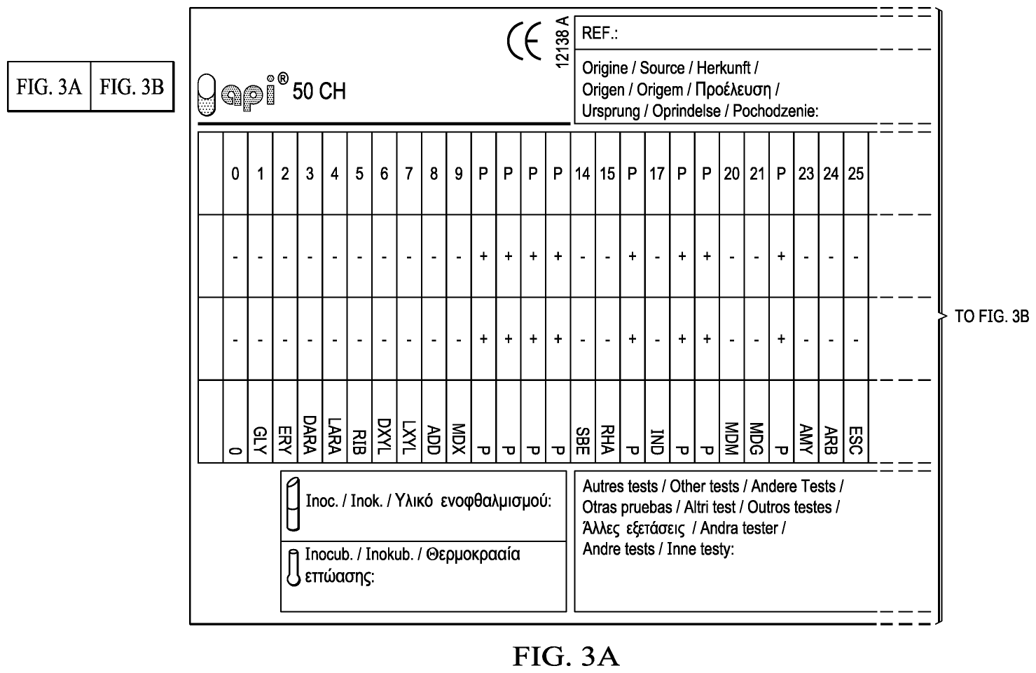

FIG. 3 is an image of the API web software results. Concludes that based on substrates above that it is Lactobacillus salivarius.

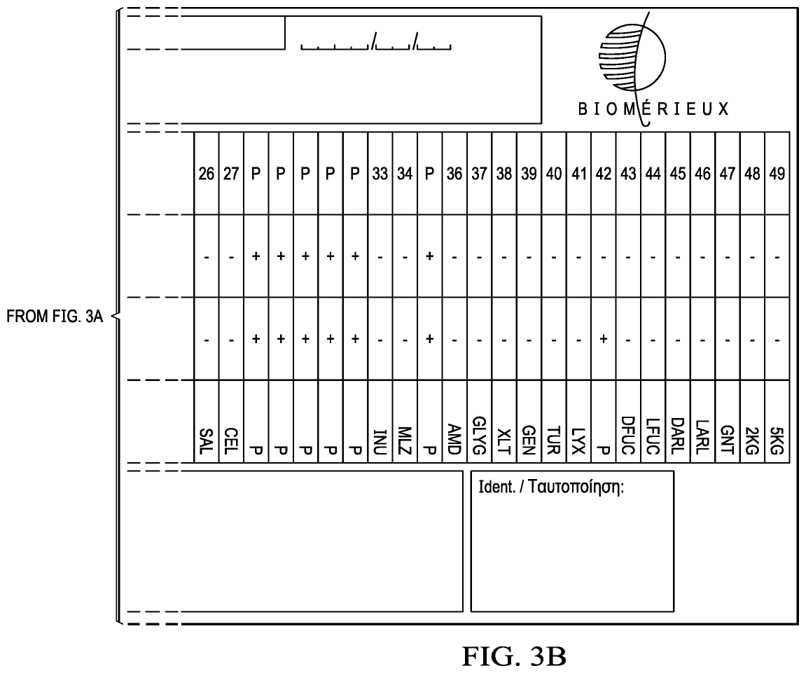

FIG. 4 is an image of a spot agar test plate. Out of hundreds of isolates screened for antimicrobial antagonism towards Salmonella, the L28 isolate showed the greatest zone of inhibition. However, the zone of inhibition did depend on the salmonella strain. Although, on average the 128 created a zone of inhibition that was approximately a 10 mm zone.

The present invention shows a reduction of Salmonella on dry kibble pet food using lactic acid bacteria L28. A collection of Lactic Acid Bacteria isolated from various food sources have shown great inhibitory activity against Salmonella when grown in co-culture conditions in laboratory media. Isolate, denoted here as L28, has led to the greatest reductions of Salmonella in vitro. L28 isolates have been shown to reduce the Listeria monocytogenes on stainless steel to undetectable levels after 24 hrs. In addition, L28 isolates have been shown to significantly reduce Salmonella in raw chicken fat after 24 hrs and brought to undetectable levels after day 3. L28 isolates have been shown to reduce significantly Salmonella on dry pet food kibble after 4 hrs and undetectable levels after 3 days.

The present invention has been shown to reduce Salmonella on a dog kibble. Salmonella cultures were prepared using three separate Salmonella (Typimurium, Enteritids, Newport) strains that were grown in Tryptic Soy Broth at 37° C. for 24 hrs. After independent strain enrichment, the strains were combines for a 3 strain cocktail. The cocktail was then aliquot, 1 ml into eppendorf tubes with 100 ml of glycerol. Cocktail culture had a final concentration of approximately log₁₀ 8.00 cfu/ml. The three strain cocktail was stored in an ultra-low −80° C. freezer.

Preparation of a lactic acid bacteria culture. L28 was enriched for 24 hrs in MRS broth at 37° C. for 24 hrs. The culture was then aliquot, 1 ml into eppendorf tubes with 100 ml of glycerol. Culture had a final concentration of approximately log₁₀ 8.00 cfu/ml. The culture was stored in an ultra low −80° C. freezer.

Concentrating lactic acid bacteria culture. Two separate one litter bottles of MRS broth were inoculated with 100 microliters of the frozen L28 culture. These two litters of MRS were enriched for 24 hrs at 37° C. To concentrate the L28 culture, the 4 conical tubes with 40 ml of the enriched L28 were centrifuged. The centrifugation parameters were set at 6000 rpm, 6 minutes at 4° C. The pelleted cells were retained and the supernatant was dumped. This process was repeated until all 2 litters were processed. The pellet was re-suspended in 5 ml of supernatant from the MRS broth. The final product yielded approximately 25 ml of concentrated Lactic Acid Bacteria culture at approximately log₁₀ 10.00 cfu/ml.

Inoculation of chicken fat. The chicken fat was provided by the commercial dog food company. Both the chicken fat control group and treatment group got 1 ml of the frozen Salmonella cocktail culture (log₁₀ 8.00 cfu/ml) added to 40 ml of chicken fat. The control chicken fat was co-inoculated with a 20 ml cell free/blank of MRS broth. The treatment chicken fat was co-inoculated with a 20 ml logᵢₙ 10.00 cfu/ml L28 culture. Thus, a final volume was 60 ml of chicken fat slurry that would be applied to dry dog food kibble.

Application on kibble. For both control group and treatment group ½ (212 grams) pound of dog food was weighed out. The 60 ml of respective chicken fat slurry was added to control and treatment. The initial concentration of Salmonella on the dry kibble at zero hour was approximately log₁₀ 6.00 cfu/g. The dry kibble was given 4 hours to dry under the hood and absorb the chicken fat slurry and facilitate attachment of salmonella to the dry kibble.

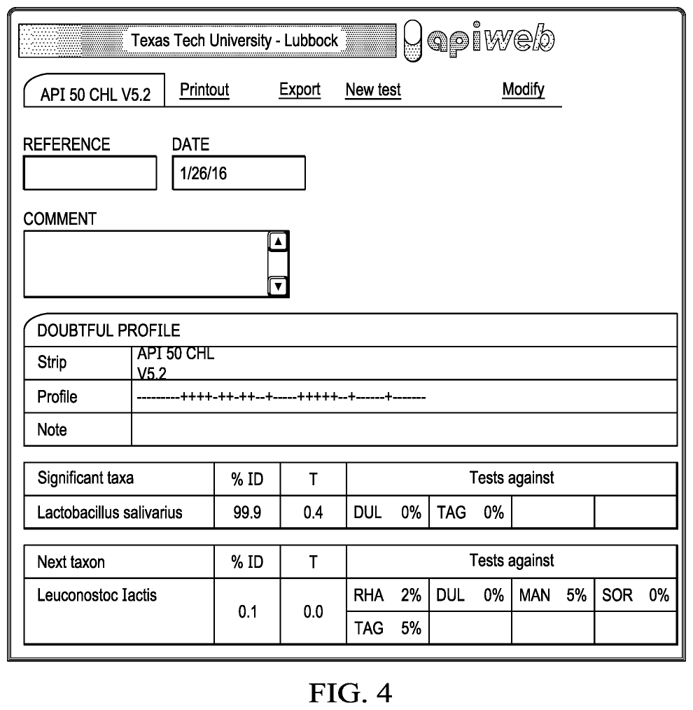

FIG. 5 shows a plot of the Reduction of Salmonella on Dry Kibble Pet Food using Novel Lactic Acid Bacteria. After 4 hrs of drying the Salmonella on the pet kibble drastically decreased in both the control and treatment groups. However, there was a much bigger decrease of Salmonella for treatment group that utilized the L28 Lactic Acid Bacteria intervention. The early reduction at 4 hrs may be due to the production of lactic acid. The culture is concentrated down and instead of re-suspending pellet in buffered peptone water it is re-suspended in its own supernatant. Thus, the method of application of L28 on kibble for these types of reductions in Salmonella may affect activity. In comparison of the control and the treatment showed approximately the same log concentration at 0 hrs; approximately 1.5 log reduction at 4 hrs; approximately 2.0 log reduction at 24 hrs; approximately 2.81 log reduction at 72 hrs. Salmonella not detected on treatment samples.

The present invention provides novel lactic acid bacteria (L14 and L28) as a biocontrol agent for inhibition of Salmonella in a raw chicken fat dog food ingredient. Chicken fat being a rich energy source has many important functions in the canine and feline diet. Salmonella is a major pathogen in poultry products and is a frequent carrier of these bacteria.

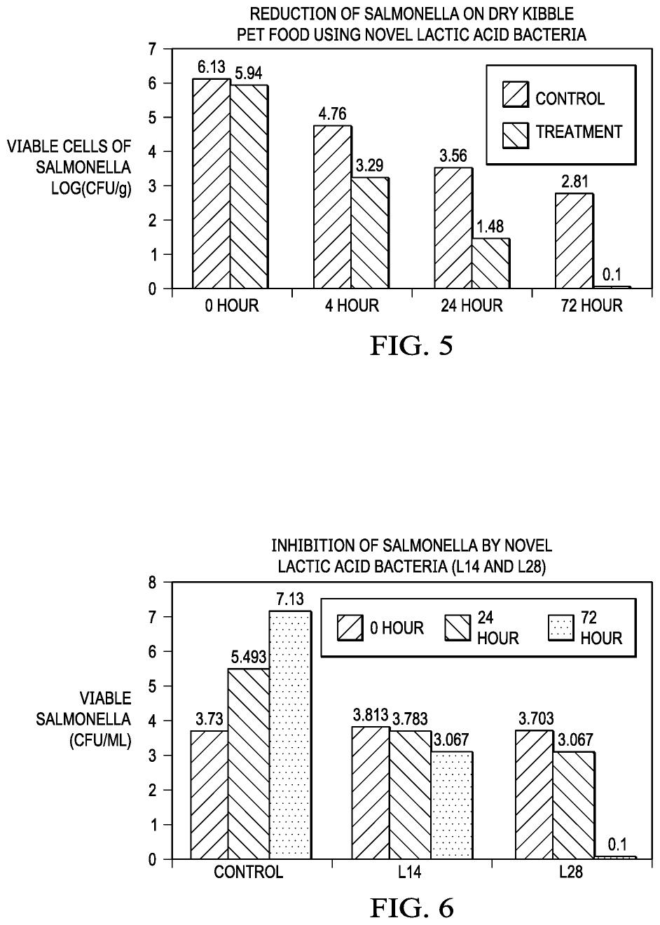

FIG. 6 is an image of the inhibition of Salmonella by lactic acid bacteria (L14, L28). The present invention provides novel isolated lactic acid bacteria (LAB)(L14, L28) that reduce the amount of Salmonella (typhimurium, enteritdis and newport) in raw chicken fat stored at room temperature. Chicken fat was provided by commercial dog food company. For both control and treatment groups, approximately 40 ml of chicken fat was inoculated with a 3-strain salmonella for a final concentration of log 3.00 cfu/ml. Each treatment group got respective treatment of (L14 or L28) for a final concentration of log 6.00 cfu/ml. The 40 ml chicken fat was aliquot by 10 ml for each time point, and enumerated on day 0, 1 and 3 on Xylose Lysine deoxycholate (XLD) agar. After day 1 there were statistical significant differences between the control and the treatments for counts of Salmonella. By day 1 and 3 the salmonella in the control chicken fat had grown to approximately log 5.49 cfu/ml and log 7.13 cfu/ml, respectively. For the L14 treatment on day 3, there was a 4.06 log reduction of Salmonella. Moreover, on day 3 for L28 treatment there was a 7.13 log reduction and not detectable by means of direct agar plating method.

Pets that consume contaminated pet kibble can be colonized with Salmonella organisms without exhibiting clinical signs, making the pet a possible source of contamination to people in the household. Lactic Acid Bacteria can inhibit Salmonella and can be provided to processors in various forms (e.g., frozen, liquid or freeze-dried) and application can be easily implemented into current operations.

The present invention also provides Lactic Acid Bacteria (L28, FS56) as bio-sanitizers to inhibit Listeria monocytogenes on stainless steel surfaces. Listeria monocytogenes is known to have the ability to attach and form biofilms on many surfaces including stainless steel. Biofilm is not easily removed with common chemical sanitizing methods used in the industry. Therefore, finding innovative ways to inhibit Listeria monocytogenes growth and biofilm formation is necessary. The present invention provides Lactic Acid Bacteria (L28) and commercially available (FS56) Lactic Acid Bacteria in inhibition of Listeria monocytogenes (N1-002) on stainless steel coupons.

Sterile stainless steel coupons (2cm×2cm) were placed into 6-well plates with 2m1 of Listeria monocytogenes (log₁₀ 5.00 cfu/ml) and incubated 24 hrs for attachment. After the 24 hrs the Listeria monocytogenes was removed and each treatment and control were added. The treatments were with strains L28, FS56 at a concentration of log₁₀ 8.00 cfu/ml and the control was with a blank of de Man, Rogosa and Sharpe (MRS) Broth. The Listeria monocytogenes counts were evaluated on modified oxford agar.

Statistical differences (P<0.05) among all of the treatments and the control for counts of Listeria monocytogenes were observed. By the end of the 24 hrs the MRS control had increased to log 5.76 cfu/cm² of Listeria monocytogenes. For the treatments, FS56 and L28 had log reduction of 3.1 cfu/cm² and 5.76 cfu/cm² respectively. The L28 Lactic Acid Bacteria was so effective that the Listeria monocytogenes was not detectable by means of direct agar plating method indicating it is more effective than the FS56 which is currently commercially available.

Animal feed compositions effective in poultry, swine, sheep, goats, and cattle are generally prepared by mixing the compounds of the present invention with a sufficient amount of animal feed to provide from about 1 to 1000 ppm of the compound in the feed. Animal feed supplements can be prepared by admixing about 75% to 95% by weight of a compound of the present invention with about 5% to about 25% by weight of a suitable carrier or diluent. Carriers suitable for use to make up the feed supplement compositions include the following: alfalfa meal, soybean meal, cottonseed oil meal, linseed oil meal, sodium chloride, cornmeal, cane molasses, urea, bone meal, corncob meal and the like. The carrier promotes a uniform distribution of the active ingredients in the finished feed into which the supplement is blended. It thus performs an important function by ensuring proper distribution of the active ingredient throughout the feed. The supplement is used as a top dressing for the feed, it likewise helps to ensure uniformity of distribution of the active material across the top of the dressed feed.

The preferred medicated swine, cattle, sheep and goat feed generally contain from 0.01 to 400 grams of active ingredient per ton of feed, the optimum amount for these animals usually being about 50 to 300 grams per ton of feed. The preferred poultry and domestic pet feed usually contain about 0.01 to 400 grams and preferably 10 to 400 grams of active ingredient per ton of feed.

Paste formulations can be prepared by dispersing the active compounds in a pharmaceutically acceptable oil such as peanut oil, sesame oil, corn oil or the like. Pellets containing an effective amount of the compounds of the present invention can be prepared by admixing the compounds of the present invention with a diluent such as carbowax, carnuba wax, and the like, and a lubricant, such as magnesium or calcium stearate, can be added to improve the pelleting process. It is, of course, recognized that more than one pellet may be administered to an animal to achieve the desired dose level which will provide the increase in lean meat deposition and improvement in lean meat to fat ratio desired. Moreover, it has been found that implants may also be made periodically during the animal treatment period in order to maintain the proper drug level in the animal's body. For the poultry and swine raisers, using the method of the present invention yields leaner animals.

Table 1 includes numerous strains that can be used with the present invention for a variety of feed, e.g., dog and cat food.

| | | Gram | | | Antimicrobial | |
|---|---|---|---|---|---|---|
| Name | Source | stain | API estimate | WGS | Resistance Genes | Virulence Factor Genes |
| MM13A | Dog | G+ | 96.9% | Lactobacillus | lnuC | (groEL) chaperonin GroEL |
| | | rods | Leuconstoc | animalis | [Streptococcus | [GroEL (CVF403)] [Clostridium |
| | | | lactis | | agalactiae] | perfringens str. 13] |
| | | | | | | (SSU98_1513) phosphopyruvate |
| | | | | | | hydratase [Fibronectin-binding |
| | | | | | | protein (AI215)] [Streptococcus |
| | | | | | | suis 98HAH33] |
| MM13B | Dog | G+ | 69.1% | Close to | msrC | (EFAU085_00344) LacI family |
| | | Cocci | Lactococcus | Enterococcus | [Enterococcus | sugar-binding transcriptional |
| | | | lactis | lactis | faecium] | regulator [BopD (CVF615)] |
| | | | | | efmA | [Enterococcus faecium |
| | | | | | [Enterococcus | Aus0085] |
| | | | | | faecium] | (tig/ropA) trigger factor [Trigger |
| | | | | | adeC | factor (CVF149)] [Streptococcus |
| | | | | | [Enterococcus | agalactiae 2603V/R] |
| | | | | | faecium DO] | (clpE) ATP-dependent protease |
| | | | | | AAC(6′)-Ii | [ClpE (VF0073)] [Listeria |
| | | | | | [Enterococcus | monocytogenes EGD-e] |
| | | | | | faecium] | (EFAU004_00491) periplasmic |
| | | | | | | solute binding protein [EfaA |
| | | | | | | (CVF610)] [Enterococcus |
| | | | | | | faecium Aus0004] |
| | | | | | | (tuf) elongation factor Tu [EF- |
| | | | | | | Tu (CVF587)] [Mycoplasma |
| | | | | | | gallisepticum str. R(low)] |
| | | | | | | (rfbA-1) glucose-1-phosphate |
| | | | | | | thymidylyltransferase [Capsule |
| | | | | | | (CVF186)] [Streptococcus |
| | | | | | | gordonii str. Challis substr. |
| | | | | | | CH1] |
| | | | | | | (rmlB) DTDP-glucose-4,6- |
| | | | | | | dehydratase, putative [Capsule |
| | | | | | | (CVF186)] [Streptococcus |
| | | | | | | sanguinis SK36] |
| | | | | | | (SSU98_1513) phosphopyruvate |
| | | | | | | hydratase [Fibronectin-binding |
| | | | | | | protein (AI215)] [Streptococcus |
| | | | | | | suis 98HAH33] |
| | | | | | | (plr/gapA) glyceraldehyde-3- |
| | | | | | | phosphate dehydrogenase, type I |
| | | | | | | [Streptococcal plasmin |
| | | | | | | receptor/GAPDH (CVF123)] |
| | | | | | | [Streptococcus pneumoniae |
| | | | | | | Hungary19A-6] |
| | | | | | | (uppS) undecaprenyl |
| | | | | | | diphosphate synthase [Capsule |
| | | | | | | (CVF618)] [Enterococcus |
| | | | | | | faecium Aus0004] |
| | | | | | | (EFAU085_01747) |
| | | | | | | phosphatidate |
| | | | | | | cytidylyltransferase [Capsule |
| | | | | | | (CVF618)] [Enterococcus |
| | | | | | | faecium Aus0085] |
| | | | | | | (sagA) SagA [Fibronectin- |
| | | | | | | binding protein (AI158)] |
| | | | | | | [Enterococcus faecium U0317] |
| | | | | | | (sgrA) cell wall anchored |
| | | | | | | protein SgrA [SgrA (VF0540)] |
| | | | | | | [Enterococcus faecium DO] |
| | | | | | | (sgrA) cell wall anchored |
| | | | | | | protein SgrA [SgrA (VF0540)] |
| | | | | | | [Enterococcus faecium DO] |
| | | | | | | (ACI49667) putative pilus tip |
| | | | | | | protein [PilB-type pili (PGS3) |
| | | | | | | (AI133)] [Enterococcus faecium |
| | | | | | | str. E1165] |
| | | | | | | (ACI49666) putative minor pilin |
| | | | | | | subunit [PilB-type pili (PGS3) |
| | | | | | | (AI133)] [Enterococcus faecium |
| | | | | | | str. E1165] |
| | | | | | | (pilB) PilB [PilB-type pili |
| | | | | | | (PGS3) (AI133)] [Enterococcus |
| | | | | | | faecium str. E1165] |
| | | | | | | (ACI49664) putative pilus- |
| | | | | | | dedicated sortase [PilB-type pili |
| | | | | | | (PGS3) (AI133)] [Enterococcus |
| | | | | | | faecium str. E1165] |
| | | | | | | (acm) collagen adhesin |
| | | | | | | precursor Acm [Acm (VF0419)] |
| | | | | | | [Enterococcus faecium str. |
| | | | | | | TX2555] |
| | | | | | | (M7W_2305) Collagen binding |
| | | | | | | protein Cna [Acm (CVF817)] |
| | | | | | | [Enterococcus faecium NRRL |
| | | | | | | B-2354] |
| | | | | | | (M7W_2305) Collagen binding |
| | | | | | | protein Cna [Acm (CVF817)] |
| | | | | | | [Enterococcus faecium NRRL |
| | | | | | | B-2354] |
| | | | | | | (bsh) putative conjugated bile |
| | | | | | | acid hydrolase [Bile-salt |
| | | | | | | hydrolase (CVF250)] [Listeria |
| | | | | | | ivanovii subsp. ivanovii PAM |
| | | | | | | 55] |
| P13 | Dog | | 99.6% | Weisella | | (EFD32_2101) phosphatidate |
| | | | Leuconostoc | paramesenteroides | | cytidylyltransferase [Capsule |
| | | | mesenteroides | | | (CVF618)] [Enterococcus |
| | | | | | | faecalis D32] |
| | | | | | | (tuf) Elongation factor Tu [EF- |
| | | | | | | Tu (CVF587)] [Mycoplasma |
| | | | | | | synoviae 53] |
| P17 | Dog | | 91.8% | Enterococcus | msrC | (plr/gapA) glyceraldehyde-3- |
| | | | Lactococcus | faecium | [Enterococcus | phosphate dehydrogenase, type I |
| | | | lactis | | faecium] | [Streptococcal plasmin |
| | | | | | adeC | receptor/GAPDH (CVF123)] |
| | | | | | [Enterococcus | [Streptococcus pneumoniae |
| | | | | | faecium DO] | Hungary 19A-6] |
| | | | | | efmA | (SSU98_1513) phosphopyruvate |
| | | | | | [Enterococcus | hydratase [Fibronectin-binding |
| | | | | | faecium] | protein (AI215)] [Streptococcus |
| | | | | | tetM [Clostridium | suis 98HAH33] |
| | | | | | difficile 630] | (sagA) SagA [Fibronectin- |
| | | | | | AAC(6′)-Ii | binding protein (AI158)] |
| | | | | | [Enterococcus | [Enterococcus faecium U0317] |
| | | | | | faecium] | (tuf) elongation factor Tu [EF- |
| | | | | | | Tu (CVF587)] [Mycoplasma |
| | | | | | | gallisepticum str. R(low)] |
| | | | | | | (tig/ropA) trigger factor [Trigger |
| | | | | | | factor (CVF149)] [Streptococcus |
| | | | | | | agalactiae 2603V/R] |
| | | | | | | (rfbA-1) glucose-1-phosphate |
| | | | | | | thymidylyltransferase [Capsule |
| | | | | | | (CVF186)] [Streptococcus |
| | | | | | | gordonii str. Challis substr. |
| | | | | | | CH1] |
| | | | | | | (clpE) ATP-dependent protease |
| | | | | | | [ClpE (VF0073)] [Listeria |
| | | | | | | monocytogenes EGD-e] |
| | | | | | | (scm) collagen-binding |
| | | | | | | MSCRAMM Scm (Fms10) |
| | | | | | | [Scm (CVF818)] [Enterococcus |
| | | | | | | faecium DO] |
| | | | | | | (lisR) two-component response |
| | | | | | | regulator [LisR/LisK (CVF253)] |
| | | | | | | [Listeria innocua Clip11262] |
| | | | | | | (EFAU08_00344) LacI family |
| | | | | | | sugar-binding transcriptional |
| | | | | | | regulator [BopD (CVF615)] |
| | | | | | | [Enterococcus faecium |
| | | | | | | Aus0085] |
| | | | | | | (EFAU085_01747) |
| | | | | | | phosphatidate cytidylyltransferase |
| | | | | | | [Capsule (CVF618)] |
| | | | | | | [Enterococcus faecium Aus0085] |
| | | | | | | (uppS) undecaprenyl |
| | | | | | | diphosphate synthase [Capsule |
| | | | | | | (CVF618)] [Enterococcus |
| | | | | | | faecium Aus0085] |
| | | | | | | (bsh) putative conjugated bile |
| | | | | | | acid hydrolase [Bile-salt |
| | | | | | | hydrolase (CVF250)] [Listeria |
| | | | | | | ivanovii subsp. ivanovii PAM |
| | | | | | | 55] |
| | | | | | | (EFAU004_00491) periplasmic |
| | | | | | | solute binding protein [EfaA |
| | | | | | | (CVF610)] [Enterococcus |
| | | | | | | faecium Aus0004] |
| | | | | | | (ACI49667) putative pilus tip |
| | | | | | | protein [PilB-type pili (PGS3) |
| | | | | | | (AI133)] [Enterococcus faecium |
| | | | | | | str. E1165] |
| | | | | | | (ACI49666) putative minor pilin |
| | | | | | | subunit [PilB-type pili (PGS3) |
| | | | | | | (AI133)] [Enterococcus faecium |
| | | | | | | str. E1165] |
| | | | | | | (pilB) PilB [PilB-type pili |
| | | | | | | (PGS3) (AI133)] [Enterococcus |
| | | | | | | faecium str. E1165] |
| | | | | | | (ACI49664) putative pilus- |
| | | | | | | dedicated sortase [PilB-type pili |
| | | | | | | (PGS3) (AI133)] [Enterococcus |
| | | | | | | faecium str. E1165] |
| | | | | | | (pilF) minor pilin subunit [PilA- |
| | | | | | | type pili (PGS1, pilin gene |
| | | | | | | clusters 1) (AI132)] |
| | | | | | | [Enterococcus faecium str. |
| | | | | | | E1165] |
| | | | | | | (ACI49669) hypothetical |
| | | | | | | hydrophobic peptide [PilA-type |
| | | | | | | pili (PGS1, pilin gene clusters 1) |
| | | | | | | (AI132)] [Enterococcus faecium |
| | | | | | | str. E1165] |
| | | | | | | (pilE) cell wall-associated |
| | | | | | | LPXTG-like protein [PilA-type |
| | | | | | | pili (PGS1, pilin gene clusters 1) |
| | | | | | | (AI132)] [Enterococcus faecium |
| | | | | | | str. E1165] |
| | | | | | | (ACI49670) putative pilus- |
| | | | | | | dedicated sortase [PilA-type pili |
| | | | | | | (PGS1, pilin gene clusters 1) |
| | | | | | | (AI132)] [Enterococcus faecium |
| | | | | | | str. E1165] |
| | | | | | | (pilA) PilA [PilA-type pili |
| | | | | | | (PGS1, pilin gene clusters 1) |
| | | | | | | (AI132)] [Enterococcus faecium |
| | | | | | | str. E1165] |
| | | | | | | (ACI49672) putative |
| | | | | | | housekeeping sortase [PilA-type |
| | | | | | | pili (PGS1, pilin gene clusters 1) |
| | | | | | | (AI132)] [Enterococcus faecium |
| | | | | | | str. E1165] |
| | | | | | | (M7W_2305) Collagen binding |
| | | | | | | protein Cna [Acm (CVF817)] |
| | | | | | | [Enterococcus faecium NRRL |
| | | | | | | B-2354] |
| | | | | | | (SGO_2024) Extracellular |
| | | | | | | polysaccharide biosynthesis |
| | | | | | | [Capsule (CVF186)] |
| | | | | | | [Streptococcus gordonii str. |
| | | | | | | Challis substr. CH1] |
| | | | | | | (bsh) putative conjugated bile |
| | | | | | | acid hydrolase [Bile-salt |
| | | | | | | hydrolase (CVF250)] [Listeria |
| | | | | | | ivanovii subsp. ivanovii PAM |
| | | | | | | 55] |
| Ecat | Cat | | 64.3% | Enterococcus | AAC(6′)-Iid | (lap) bifunctional aldehyde- |
| | | | lactococcus | hirae | [Enterococcus | alcohol dehydrogenase [Listeria |
| | | | lactis | | hirae] | adhesion protein (CVF228)] |
| | | | | | tetL [Geobacillus | [Listeria monocytogenes |
| | | | | | stearothermophilus] | SLCC2755] |
| | | | | | tetM [Clostridium | (capD) capsular polysaccharide |
| | | | | | difficile 630] | synthesis enzyme [Capsule |
| | | | | | | (CVF110)] [Staphylococcus |
| | | | | | | aureus subsp. aureus MRSA252] |
| | | | | | | (EFD32_2606) polysaccharide |
| | | | | | | lyase family protein |
| | | | | | | [Hyaluronidase (CVF614)] |
| | | | | | | [Enterococcus faecalis D32] |
| | | | | | | (tuf) elongation factor Tu [EF- |
| | | | | | | Tu (CVF587)] [Mycoplasma |
| | | | | | | hyopneumoniae J] |
| | | | | | | (M3Q_285) nucleoside- |
| | | | | | | diphosphate sugar epimerase |
| | | | | | | [Capsule (CVF775)] |
| | | | | | | [Acinetobacter baumannii |
| | | | | | | TYTH-1] |
| | | | | | | (groEL) chaperonin GroEL |
| | | | | | | [GroEL (CVF403)] [Clostridium |
| | | | | | | beijerinckii NCIMB 8052] |
| | | | | | | (tuf) elongation factor Tu |
| | | | | | | [Fibronectin-binding protein |
| | | | | | | (AI171)] [Mycoplasma |
| | | | | | | pneumoniae M129] |
| | | | | | | (sagA) SagA [Fibronectin- |
| | | | | | | binding protein (AI158)] |
| | | | | | | [Enterococcus faecium U0317] |
| | | | | | | (plr/gapA) Glyceraldehyde 3- |
| | | | | | | phosphate dehydrogenase, |
| | | | | | | putative [Streptococcal plasmin |
| | | | | | | receptor/GAPDH (CVF123)] |
| | | | | | | [Streptococcus sanguinis SK36] |
| | | | | | | (eno) phosphopyruvate |
| | | | | | | hydratase [Streptococcal enolase |
| | | | | | | (CVF153)] [Streptococcus |
| | | | | | | pneumoniae D39] |
| | | | | | | (uppS) undecaprenyl |
| | | | | | | diphosphate synthase [Capsule |
| | | | | | | (CVF618)] [Enterococcus |
| | | | | | | faecium Aus0004] |
| J19 | Cabbage | G+ | ? | Enterococcus | msrC | (plr/gapA) glyceraldehyde-3- |
| | | cocci | | faecium | [Enterococcus | phosphate dehydrogenase, type I |
| | | | | | faecium] | [Streptococcal plasmin |
| | | | | | AAC(6′)-Ii | receptor/GAPDH (CVF123)] |
| | | | | | [Enterococcus | [Streptococcus pneumoniae |
| | | | | | faecium] | Hungary 19A-6] |
| | | | | | efmA | (SSU98_1513) phosphopyruvate |
| | | | | | [Enterococcus | hydratase [Fibronectin-binding |
| | | | | | faecium] | protein (AI215)] [Streptococcus |
| | | | | | adeC | suis 98HAH33] |
| | | | | | [Enterococcus | (uppS) undecaprenyl |
| | | | | | faecium DO] | diphosphate synthase [Capsule |
| | | | | | | (CVF618)] [Enterococcus |
| | | | | | | faecium Aus0085] |
| | | | | | | (EFAU085_01747) |
| | | | | | | phosphatidate |
| | | | | | | cytidylyltransferase [Capsule |
| | | | | | | (CVF618)] [Enterococcus |
| | | | | | | faecium Aus0085] |
| | | | | | | (tig/ropA) trigger factor [Trigger |
| | | | | | | factor (CVF149)] [Streptococcus |
| | | | | | | agalactiae 2603V/R] |
| | | | | | | (sagA) SagA [Fibronectin- |
| | | | | | | binding protein (AI158)] |
| | | | | | | [Enterococcus faecium U0317] |
| | | | | | | (scm) collagen-binding |
| | | | | | | MSCRAMM Scm (Fms10) |
| | | | | | | [Scm (CVF818)] [Enterococcus |
| | | | | | | faecium DO] |
| | | | | | | (scm) collagen-binding |
| | | | | | | MSCRAMM Scm (Fms10) |
| | | | | | | [Scm (CVF818)] [Enterococcus |
| | | | | | | faecium DO] |
| | | | | | | (clpE) ATP-dependent protease |
| | | | | | | [ClpE (VF0073)] [Listeria |
| | | | | | | monocytogenes EGD-e] |
| | | | | | | (EFAU085_00344) LacI family |
| | | | | | | sugar-binding transcriptional |
| | | | | | | regulator [BopD (CVF615)] |
| | | | | | | [Enterococcus faecium |
| | | | | | | Aus0085] |
| | | | | | | (lisR) two-component response |
| | | | | | | regulator [LisR/LisK (CVF253)] |
| | | | | | | [Listeria innocua Clip1 1262] |
| | | | | | | (ACI49672) putative |
| | | | | | | housekeeping sortase [PilA-type |
| | | | | | | pili (PGS1, pilin gene clusters 1) |
| | | | | | | (AI132)] [Enterococcus faecium |
| | | | | | | str. E1165] |
| | | | | | | (pilA) PilA [PilA-type pili |
| | | | | | | (PGS1, pilin gene clusters 1) |
| | | | | | | (AI132)] [Enterococcus faecium |
| | | | | | | str. E1165] |
| | | | | | | (ACI49670) putative pilus- |
| | | | | | | dedicated sortase [PilA-type pili |
| | | | | | | (PGS1, pilin gene clusters 1) |
| | | | | | | (AI132)] [Enterococcus faecium |
| | | | | | | str. E1165] |
| | | | | | | (pilE) cell wall-associated |
| | | | | | | LPXTG-like protein [PilA-type |
| | | | | | | pili (PGS1, pilin gene clusters 1) |
| | | | | | | (AI132)] [Enterococcus faecium |
| | | | | | | str. E1165] |
| | | | | | | (ACI49669) hypothetical |
| | | | | | | hydrophobic peptide [PilA-type |
| | | | | | | pili (PGS1, pilin gene clusters 1) |
| | | | | | | (AI132)] [Enterococcus faecium |
| | | | | | | str. E1165] |
| | | | | | | (pilF) minor pilin subunit [PilA- |
| | | | | | | type pili (PGS1, pilin gene |
| | | | | | | clusters 1) (AI132)] |
| | | | | | | [Enterococcus faecium str. |
| | | | | | | E1165] |
| | | | | | | (tuf) elongation factor Tu [EF- |
| | | | | | | Tu (CVF587)] [Mycoplasma |
| | | | | | | gallisepticum str. R(low)] |
| | | | | | | (sgrA) cell wall anchored |
| | | | | | | protein SgrA [SgrA (VF0540)] |
| | | | | | | [Enterococcus faecium DO] |
| | | | | | | (EFAU004_00491) periplasmic |
| | | | | | | solute binding protein [EfaA |
| | | | | | | (CVF610)] [Enterococcus |
| | | | | | | faecium Aus0004] |
| | | | | | | (bsh) putative conjugated bile |
| | | | | | | acid hydrolase [Bile-salt |
| | | | | | | hydrolase (CVF250)] [Listeria |
| | | | | | | ivanovii subsp. ivanovii PAM |
| | | | | | | 55] |
| | | | | | | (rfbA-1) glucose-1-phosphate |
| | | | | | | thymidylyltransferase [Capsule |
| | | | | | | (CVF186)] [Streptococcus |
| | | | | | | gordonii str. Challis substr. |
| | | | | | | CH1] |
| | | | | | | (ACI49664) putative pilus- |
| | | | | | | dedicated sortase [PilB-type pili |
| | | | | | | (PGS3) (AI133)] [Enterococcus |
| | | | | | | faecium str. E1165] |
| | | | | | | (pilB) PilB [PilB-type pili |
| | | | | | | (PGS3) (AI133)] [Enterococcus |
| | | | | | | faecium str. E1165] |
| | | | | | | (ACI49667) putative pilus tip |
| | | | | | | protein [PilB-type pili (PGS3) |
| | | | | | | (AI133)] [Enterococcus faecium |
| | | | | | | str. E1165] |
| | | | | | | (bsh) putative conjugated bile |
| | | | | | | acid hydrolase [Bile-salt |
| | | | | | | hydrolase (CVF250)] [Listeria |
| | | | | | | ivanovii subsp. ivanovii PAM |
| | | | | | | 55] |
| J27 | Grapes | G+ | ? | Enterococcus | msrC | (plr/gapA) glyceraldehyde-3- |
| | | cocci | | faecium | [Enterococcus | phosphate dehydrogenase, type I |
| | | | | | faecium] | [Streptococcal plasmin |
| | | | | | AAC(6′)-Ii | receptor/GAPDH (CVF123)] |
| | | | | | [Enterococcus | [Streptococcus pneumoniae |
| | | | | | faecium] | Hungary 19A-6] |
| | | | | | efmA | (SSU98_1513) phosphopyruvate |
| | | | | | [Enterococcus | hydratase [Fibronectin-binding |
| | | | | | faecium] | protein (AI215)] [Streptococcus |
| | | | | | adeC | suis 98HAH33] |
| | | | | | [Enterococcus | (uppS) undecaprenyl |
| | | | | | faecium DO] | diphosphate synthase [Capsule |
| | | | | | | (CVF618)] [Enterococcus |
| | | | | | | faecium Aus0085] |
| | | | | | | (EFAU085_01747) |
| | | | | | | phosphatidate |
| | | | | | | cytidylyltransferase [Capsule |
| | | | | | | (CVF618)] [Enterococcus |
| | | | | | | faecium Aus0085] |
| | | | | | | (tig/ropA) trigger factor [Trigger |
| | | | | | | factor (CVF149)] [Streptococcus |
| | | | | | | agalactiae 2603V/R] |
| | | | | | | (sagA) SagA [Fibronectin- |
| | | | | | | binding protein (AI158)] |
| | | | | | | [Enterococcus faecium U0317] |
| | | | | | | (scm) collagen-binding |
| | | | | | | MSCRAMM Scm (Fms10) |
| | | | | | | [Scm (CVF818)] [Enterococcus |
| | | | | | | faecium DO] |
| | | | | | | (scm) collagen-binding |
| | | | | | | MSCRAMM Scm (Fms10) |
| | | | | | | [Scm (CVF818)] [Enterococcus |
| | | | | | | faecium DO] |
| | | | | | | (clpE) ATP-dependent protease |
| | | | | | | [ClpE (VF0073)] [Listeria |
| | | | | | | monocytogenes EGD-e] |
| | | | | | | (ACI49667) putative pilus tip |
| | | | | | | protein [PilB-type pili (PGS3) |
| | | | | | | (AI133)] [Enterococcus faecium |
| | | | | | | str. E1165] |
| | | | | | | (pilB) PilB [PilB-type pili |
| | | | | | | (PGS3) (AI133)] [Enterococcus |
| | | | | | | faecium str. E1165] |
| | | | | | | (ACI49664) putative pilus- |
| | | | | | | dedicated sortase [PilB-type pili |
| | | | | | | (PGS3) (AI133)] [Enterococcus |
| | | | | | | faecium str. E1165] |
| | | | | | | (EFAU085_00344) LacI family |
| | | | | | | sugar-binding transcriptional |
| | | | | | | regulator [BopD (CVF615)] |
| | | | | | | [Enterococcus faecium |
| | | | | | | Aus0085] |
| | | | | | | (pilF) minor pilin subunit [PilA- |
| | | | | | | type pili (PGS1, pilin gene |
| | | | | | | clusters 1) (AI132)] |
| | | | | | | [Enterococcus faecium str. |
| | | | | | | E1165] |
| | | | | | | (ACI49669) hypothetical |
| | | | | | | hydrophobic peptide [PilA-type |
| | | | | | | pili (PGS1, pilin gene clusters 1) |
| | | | | | | (AI132)] [Enterococcus faecium |
| | | | | | | str. E1165] |
| | | | | | | (pilE) cell wall-associated |
| | | | | | | LPXTG-like protein [PilA-type |
| | | | | | | pili (PGS1, pilin gene clusters 1) |
| | | | | | | (AI132)] [Enterococcus faecium |
| | | | | | | str. E1165] |
| | | | | | | (ACI49670) putative pilus- |
| | | | | | | dedicated sortase [PilA-type pili |
| | | | | | | (PGS1, pilin gene clusters 1) |
| | | | | | | (AI132)] [Enterococcus faecium |
| | | | | | | str. E1165] |
| | | | | | | (pilA) PilA [PilA-type pili |
| | | | | | | (PGS1, pilin gene clusters 1) |
| | | | | | | (AI132)] [Enterococcus faecium |
| | | | | | | str. E1165] |
| | | | | | | (ACI49672) putative |
| | | | | | | housekeeping sortase [PilA-type |
| | | | | | | pili (PGS1, pilin gene clusters 1) |
| | | | | | | (AI132)] [Enterococcus faecium |
| | | | | | | str. E1165] |
| | | | | | | (tuf) elongation factor Tu [EF- |
| | | | | | | Tu (CVF587)] [Mycoplasma |
| | | | | | | gallisepticum str. R(low)] |
| | | | | | | (EFAU004_00491) periplasmic |
| | | | | | | solute binding protein [EfaA |
| | | | | | | (CVF610)] [Enterococcus |
| | | | | | | faecium Aus0004] |
| | | | | | | (sgrA) cell wall anchored |
| | | | | | | protein SgrA [SgrA (VF0540)] |
| | | | | | | [Enterococcus faecium DO] |
| | | | | | | (rfbA-1) glucose-1-phosphate |
| | | | | | | thymidylyltransferase [Capsule |
| | | | | | | (CVF186)] [Streptococcus |
| | | | | | | gordonii str. Challis substr. |
| | | | | | | CH1] |
| | | | | | | (bsh) putative conjugated bile |
| | | | | | | acid hydrolase [Bile-salt |
| | | | | | | hydrolase (CVF250)] [Listeria |
| | | | | | | ivanovii subsp. ivanovii PAM |
| | | | | | | 55] |
| | | | | | | (lisR) two-component response |
| | | | | | | regulator [LisR/LisK (CVF253)] |
| | | | | | | [Listeria innocua Clip1 1262] |
| J35 | Tofu | G+ | ? | Enterococcus | AAC(6′)-Ii | (plr/gapA) glyceraldehyde-3- |
| | | cocci | | faecium | [Enterococcus | phosphate dehydrogenase, type I |
| | | | | | faecium] | [Streptococcal plasmin |
| | | | | | efmA | receptor/GAPDH (CVF123)] |
| | | | | | [Enterococcus | [Streptococcus pneumoniae |
| | | | | | faecium] | Hungary 19A-6] |
| | | | | | adeC | (SSU98_1513) phosphopyruvate |
| | | | | | [Enterococcus | hydratase [Fibronectin-binding |
| | | | | | faecium DO] | protein (AI215)] [Streptococcus |
| | | | | | msrC | suis 98HAH33] |
| | | | | | [Enterococcus | (EFAU085_00344) LacI family |
| | | | | | faecium] | sugar-binding transcriptional |
| | | | | | | regulator [BopD (CVF615)] |
| | | | | | | [Enterococcus faecium |
| | | | | | | Aus0085] |
| | | | | | | (tuf) elongation factor Tu [EF- |
| | | | | | | Tu (CVF587)] [Mycoplasma |
| | | | | | | gallisepticum str. R(low)] |
| | | | | | | (scm) collagen-binding |
| | | | | | | MSCRAMM Scm (Fms10) |
| | | | | | | [Scm (CVF818)] [Enterococcus |
| | | | | | | faecium DO] |
| | | | | | | (scm) collagen-binding |
| | | | | | | MSCRAMM Scm (Fms10) |
| | | | | | | [Scm (CVF818)] [Enterococcus |
| | | | | | | faecium DO] |
| | | | | | | (clpE) ATP-dependent protease |
| | | | | | | [ClpE (VF0073)] [Listeria |
| | | | | | | monocytogenes EGD-e] |
| | | | | | | (sagA) SagA [Fibronectin- |
| | | | | | | binding protein (AI158)] |
| | | | | | | [Enterococcus faecium U0317] |
| | | | | | | (pilF) minor pilin subunit [PilA- |
| | | | | | | type pili (PGS1, pilin gene |
| | | | | | | clusters 1) (AI132)] |
| | | | | | | [Enterococcus faecium str. |
| | | | | | | E1165] |
| | | | | | | (ACI49669) hypothetical |
| | | | | | | hydrophobic peptide [PilA-type |
| | | | | | | pili (PGS1, pilin gene clusters 1) |
| | | | | | | (AI132)] [Enterococcus faecium |
| | | | | | | str. E1165] |
| | | | | | | (pilE) cell wall-associated |
| | | | | | | LPXTG-like protein [PilA-type |
| | | | | | | pili (PGS1, pilin gene clusters 1) |
| | | | | | | (AI132)] [Enterococcus faecium |
| | | | | | | str. E1165] |
| | | | | | | (ACI49670) putative pilus- |
| | | | | | | dedicated sortase [PilA-type pili |
| | | | | | | (PGS1, pilin gene clusters 1) |
| | | | | | | (AI132)] [Enterococcus faecium |
| | | | | | | str. E1165] |
| | | | | | | (pilA) PilA [PilA-type pili |
| | | | | | | (PGS1, pilin gene clusters 1) |
| | | | | | | (AI132)] [Enterococcus faecium |
| | | | | | | str. E1165] |
| | | | | | | (ACI49672) putative |
| | | | | | | housekeeping sortase [PilA-type |
| | | | | | | pili (PGS1, pilin gene clusters 1) |
| | | | | | | (AI132)] [Enterococcus faecium |
| | | | | | | str. E1165] |
| | | | | | | (EFAU004_00491) periplasmic |
| | | | | | | solute binding protein [EfaA |
| | | | | | | (CVF610)] [Enterococcus |
| | | | | | | faecium Aus0004] |
| | | | | | | (tig/ropA) trigger factor [Trigger |
| | | | | | | factor (CVF149)] [Streptococcus |
| | | | | | | agalactiae 2603V/R] |
| | | | | | | (rfbA-1) glucose-1-phosphate |
| | | | | | | thymidylyltransferase [Capsule |
| | | | | | | (CVF186)] [Streptococcus |
| | | | | | | gordonii str. Challis substr. |
| | | | | | | CH1] |
| | | | | | | (bsh) putative conjugated bile |
| | | | | | | acid hydrolase [Bile-salt |
| | | | | | | hydrolase (CVF250)] [Listeria |
| | | | | | | ivanovii subsp. ivanovii PAM |
| | | | | | | 55] |
| | | | | | | (uppS) undecaprenyl |
| | | | | | | diphosphate synthase [Capsule |
| | | | | | | (CVF618)] [Enterococcus |
| | | | | | | faecium Aus0085] |
| | | | | | | (EFAU085_01747) |
| | | | | | | phosphatidate |
| | | | | | | cytidylyltransferase [Capsule |
| | | | | | | (CVF618)] [Enterococcus |
| | | | | | | faecium Aus0085] |
| | | | | | | (ACI49664) putative pilus- |
| | | | | | | dedicated sortase [PilB-type pili |
| | | | | | | (PGS3) (AI133)] [Enterococcus |
| | | | | | | faecium str. E1165] |
| | | | | | | (pilB) PilB [PilB-type pili |
| | | | | | | (PGS3) (AI133)] [Enterococcus |
| | | | | | | faecium str. E1165] |
| | | | | | | (ACI49667) putative pilus tip |
| | | | | | | protein [PilB-type pili (PGS3) |
| | | | | | | (AI133)] [Enterococcus faecium |
| | | | | | | str. E1165] |
| | | | | | | (rmlC) dTDP-4-keto-L- |
| | | | | | | rhamnose reductase [Capsule |
| | | | | | | (CVF186)] [Streptococcus |
| | | | | | | thermophilus LMG 18311] |
| | | | | | | (lisR) two-component response |
| | | | | | | regulator [LisR/LisK (CVF253)] |
| | | | | | | [Listeria innocua Clip1 1262] |
| | | | | | | (sgrA) cell wall anchored |
| | | | | | | protein SgrA [SgrA (VF0540)] |
| | | | | | | [Enterococcus faecium DO] |
| J43 | Carrot | G+ | ? | Enterococcus | msrC | (SSU98_1513) phosphopyruvate |
| | | cocci | | faecium | [Enterococcus | hydratase [Fibronectin-binding |
| | | | | | faecium] | protein (AI215)] [Streptococcus |
| | | | | | AAC(6′)-Ii | suis 98HAH33] |
| | | | | | [Enterococcus | (plr/gapA) glyceraldehyde-3- |
| | | | | | faecium] | phosphate dehydrogenase, type I |
| | | | | | efmA | [Streptococcal plasmin |
| | | | | | [Enterococcus | receptor/GAPDH (CVF123)] |
| | | | | | faecium] | [Streptococcus pneumoniae |
| | | | | | adeC | Hungary 19A-6] |
| | | | | | [Enterococcus | (ACI49667) putative pilus tip |
| | | | | | faecium DO] | protein [PilB-type pili (PGS3) |
| | | | | | | (AI133)] [Enterococcus faecium |
| | | | | | | str. E1165] |
| | | | | | | (pilB) PilB [PilB-type pili |
| | | | | | | (PGS3) (AI133)] [Enterococcus |
| | | | | | | faecium str. E1165] |
| | | | | | | (ACI49664) putative pilus- |
| | | | | | | dedicated sortase [PilB-type pili |
| | | | | | | (PGS3) (AI133)] [Enterococcus |
| | | | | | | faecium str. E1165] |
| | | | | | | (uppS) undecaprenyl |
| | | | | | | diphosphate synthase [Capsule |
| | | | | | | (CVF618)] [Enterococcus |
| | | | | | | faecium Aus0085] |
| | | | | | | (EFAU085_01747) |
| | | | | | | phosphatidate |
| | | | | | | cytidylyltransferase [Capsule |
| | | | | | | (CVF618)] [Enterococcus |
| | | | | | | faecium Aus0085] |
| | | | | | | (tig/ropA) trigger factor [Trigger |
| | | | | | | factor (CVF149)] [Streptococcus |
| | | | | | | agalactiae 2603V/R] |
| | | | | | | (sagA) SagA [Fibronectin- |
| | | | | | | binding protein (AI158)] |
| | | | | | | [Enterococcus faecium U0317] |
| | | | | | | (scm) collagen-binding |
| | | | | | | MSCRAMM Scm (Fms10) |
| | | | | | | [Scm (CVF818)] [Enterococcus |
| | | | | | | faecium DO] |
| | | | | | | (scm) collagen-binding |
| | | | | | | MSCRAMM Scm (Fms10) |
| | | | | | | [Scm (CVF818)] [Enterococcus |
| | | | | | | faecium DO] |
| | | | | | | (clpE) ATP-dependent protease |
| | | | | | | [ClpE (VF0073)] [Listeria |
| | | | | | | monocytogenes EGD-e] |
| | | | | | | (EFAU085_00344) LacI family |
| | | | | | | sugar-binding transcriptional |
| | | | | | | regulator [BopD (CVF615)] |
| | | | | | | [Enterococcus faecium |
| | | | | | | Aus0085] |
| | | | | | | (lisR) two-component response |
| | | | | | | regulator [LisR/LisK (CVF253)] |
| | | | | | | [Listeria innocua Clip1 1262] |
| | | | | | | (ACI49672) putative |
| | | | | | | housekeeping sortase [PilA-type |
| | | | | | | pili (PGS1, pilin gene clusters 1) |
| | | | | | | (AI132)] [Enterococcus faecium |
| | | | | | | str. E1165] |
| | | | | | | (pilA) PilA [PilA-type pili |
| | | | | | | (PGS1, pilin gene clusters 1) |
| | | | | | | (AI132)] [Enterococcus faecium |
| | | | | | | str. E1165] |
| | | | | | | (ACI49670) putative pilus- |
| | | | | | | dedicated sortase [PilA-type pili |
| | | | | | | (PGS1, pilin gene clusters 1) |
| | | | | | | (AI132)] [Enterococcus faecium |
| | | | | | | str. E1165] |
| | | | | | | (pilE) cell wall-associated |
| | | | | | | LPXTG-like protein [PilA-type |
| | | | | | | pili (PGS1, pilin gene clusters 1) |
| | | | | | | (AI132)] [Enterococcus faecium |
| | | | | | | str. E1165] |
| | | | | | | (ACI49669) hypothetical |
| | | | | | | hydrophobic peptide [PilA-type |
| | | | | | | pili (PGS1, pilin gene clusters 1) |
| | | | | | | (AI132)] [Enterococcus faecium |
| | | | | | | str. E1165] |
| | | | | | | (pilF) minor pilin subunit [PilA- |
| | | | | | | type pili (PGS1, pilin gene |
| | | | | | | clusters 1) (AI132)] |
| | | | | | | [Enterococcus faecium str. |
| | | | | | | E1165] |
| | | | | | | (tuf) elongation factor Tu [EF- |
| | | | | | | Tu (CVF587)] [Mycoplasma |
| | | | | | | gallisepticum str. R(low)] |
| | | | | | | (EFAU004_00491) periplasmic |
| | | | | | | solute binding protein [EfaA |
| | | | | | | (CVF610)] [Enterococcus |
| | | | | | | faecium Aus0004] |
| | | | | | | (rfbA-1) glucose-1-phosphate |
| | | | | | | thymidylyltransferase [Capsule |
| | | | | | | (CVF186)] [Streptococcus |
| | | | | | | gordonii str. Challis substr. |
| | | | | | | CH1] |
| | | | | | | (bsh) putative conjugated bile |
| | | | | | | acid hydrolase [Bile-salt |
| | | | | | | hydrolase (CVF250)] [Listeria |
| | | | | | | ivanovii subsp. ivanovii PAM |
| | | | | | | 55] |
| | | | | | | (sgrA) cell wall anchored |
| | | | | | | protein SgrA [SgrA (VF0540)] |
| | | | | | | [Enterococcus faecium DO] |
| L14 | Ground | G+ | 84.4% | Enterococcus | AAC(6′)-Iid | (groEL) chaperonin GroEL |
| | Beef | cocci | Lactobacillus | hirae | [Enterococcus | [GroEL (CVF403)] [Clostridium |
| | | | acidopholus | | hirae] | beijerinckii NCIMB 8052] |
| | | | | | | (tuf) elongation factor Tu |
| | | | | | | [Fibronectin-binding protein |
| | | | | | | (AI171)] [Mycoplasma |
| | | | | | | pneumoniae M129] |
| | | | | | | (eno) phosphopyruvate |
| | | | | | | hydratase [Streptococcal enolase |
| | | | | | | (CVF153)] [Streptococcus |
| | | | | | | pneumoniae D39] |
| | | | | | | (plr/gapA) Glyceraldehyde 3- |
| | | | | | | phosphate dehydrogenase, |
| | | | | | | putative [Streptococcal plasmin |
| | | | | | | receptor/GAPDH (CVF123)] |
| | | | | | | [Streptococcus sanguinis SK36] |
| | | | | | | (capD) capsular polysaccharide |
| | | | | | | synthesis enzyme [Capsule |
| | | | | | | (CVF110)] [Staphylococcus |
| | | | | | | aureus subsp. aureus MRSA252] |
| | | | | | | (M3Q_285) nucleoside- |
| | | | | | | diphosphate sugar epimerase |
| | | | | | | [Capsule (CVF775)] |
| | | | | | | [Acinetobacter baumannii |
| | | | | | | TYTH-1] |
| | | | | | | (tuf) elongation factor Tu [EF- |
| | | | | | | Tu (CVF587)] [Mycoplasma |
| | | | | | | hyopneumoniae J] |
| | | | | | | (EFD32_2606) polysaccharide |
| | | | | | | lyase family protein |
| | | | | | | [Hyaluronidase (CVF614)] |
| | | | | | | [Enterococcus faecalis D32] |
| | | | | | | (uppS) undecaprenyl |
| | | | | | | diphosphate synthase [Capsule |
| | | | | | | (CVF618)] [Enterococcus |
| | | | | | | faecium Aus0004] |
| | | | | | | (ACI49672) putative |
| | | | | | | housekeeping sortase [PilA-type |
| | | | | | | pili (PGS1, pilin gene clusters 1) |
| | | | | | | (AI132)] [Enterococcus faecium |
| | | | | | | str. E1165] |
| | | | | | | (pilA) PilA [PilA-type pili |
| | | | | | | (PGS1, pilin gene clusters 1) |
| | | | | | | (AI132)] [Enterococcus faecium |
| | | | | | | str. E1165] |
| | | | | | | (ACI49670) putative pilus- |
| | | | | | | dedicated sortase [PilA-type pili |
| | | | | | | (PGS1, pilin gene clusters 1) |
| | | | | | | (AI132)] [Enterococcus faecium |
| | | | | | | str. E1165] |
| | | | | | | (pilE) cell wall-associated |
| | | | | | | LPXTG-like protein [PilA-type |
| | | | | | | pili (PGS1, pilin gene clusters 1) |
| | | | | | | (AI132)] [Enterococcus faecium |
| | | | | | | str. E1165] |
| | | | | | | (ACI49669) hypothetical |
| | | | | | | hydrophobic peptide [PilA-type |
| | | | | | | pili (PGS1, pilin gene clusters 1) |
| | | | | | | (AI132)] [Enterococcus faecium |
| | | | | | | str. E1165] |
| | | | | | | (pilF) minor pilin subunit [PilA- |
| | | | | | | type pili (PGS1, pilin gene |
| | | | | | | clusters 1) (AI132)] |
| | | | | | | [Enterococcus faecium str. |
| | | | | | | E1165] |
| | | | | | | (sagA) SagA [Fibronectin- |
| | | | | | | binding protein (AI158)] |
| | | | | | | [Enterococcus faecium U0317] |
| | | | | | | (lap) bifunctional aldehyde- |
| | | | | | | alcohol dehydrogenase [Listeria |
| | | | | | | adhesion protein (CVF228)] |
| | | | | | | [Listeria monocytogenes |
| | | | | | | SLCC2755] |
| | | | | | | (SGO_2024) Extracellular |
| | | | | | | polysaccharide biosynthesis |
| | | | | | | [Capsule (CVF186)] |
| | | | | | | [Streptococcus gordonii str. |
| | | | | | | Challis substr. CH1] |
| L15 | Ground | G+ | ? | Lactobacillus | | (SSU98_1513) phosphopyruvate |
| | Beef | rods | | sakei | | hydratase [Fibronectin-binding |
| | | | | | | protein (AI215)] [Streptococcus |
| | | | | | | suis 98HAH33] |
| | | | | | | (rfbB-1) dTDP-glucose 4,6- |
| | | | | | | dehydratase [Capsule |
| | | | | | | (CVF186)] [Streptococcus |
| | | | | | | gordonii str. Challis substr. |
| | | | | | | CH1] |
| L17 | Ground | G+ | ? | Lactobacillus | | (SSU98_1513) phosphopyruvate |
| | Beef | rods | | sakei | | hydratase [Fibronectin-binding |
| | | | | | | protein (AI215)] [Streptococcus |
| | | | | | | suis 98HAH33] |
| | | | | | | (rfbB-1) dTDP-glucose 4,6- |
| | | | | | | dehydratase [Capsule |
| | | | | | | (CVF186)] [Streptococcus |
| | | | | | | gordonii str. Challis substr. |
| | | | | | | CH1] |
| L19 | Chicken | | ? | Enterococcus | msrC | (plr/gapA) glyceraldehyde-3- |
| | legs | | | faecium | [Enterococcus | phosphate dehydrogenase, type I |
| | | | | | faecium] | [Streptococcal plasmin |
| | | | | | AAC(6′)-Ii | receptor/GAPDH (CVF123)] |
| | | | | | [Enterococcus | [Streptococcus pneumoniae |
| | | | | | faecium] | Hungary 19A-6] |
| | | | | | adeC | (SSU98_1513) phosphopyruvate |
| | | | | | [Enterococcus | hydratase [Fibronectin-binding |
| | | | | | faecium DO] | protein (AI215)] [Streptococcus |
| | | | | | efmA | suis 98HAH33] |
| | | | | | [Enterococcus | (sagA) SagA [Fibronectin- |
| | | | | | faecium] | binding protein (AI158)] |
| | | | | | | [Enterococcus faecium U0317] |
| | | | | | | (scm) collagen-binding |
| | | | | | | MSCRAMM Scm (Fms10) |
| | | | | | | [Scm (CVF818)] [Enterococcus |
| | | | | | | faecium DO] |
| | | | | | | (scm) collagen-binding |
| | | | | | | MSCRAMM Scm (Fms10) |
| | | | | | | [Scm (CVF818)] [Enterococcus |
| | | | | | | faecium DO] |
| | | | | | | (clpE) ATP-dependent protease |
| | | | | | | [ClpE (VF0073)] [Listeria |
| | | | | | | monocytogenes EGD-e] |
| | | | | | | (EFAU085_00344) LacI family |
| | | | | | | sugar-binding transcriptional |
| | | | | | | regulator [BopD (CVF615)] |
| | | | | | | [Enterococcus faecium |
| | | | | | | Aus0085] |
| | | | | | | (uppS) undecaprenyl |
| | | | | | | diphosphate synthase [Capsule |
| | | | | | | (CVF618)] [Enterococcus |
| | | | | | | faecium Aus0085] |
| | | | | | | (EFAU085_01747) |
| | | | | | | phosphatidate |
| | | | | | | cytidylyltransferase [Capsule |
| | | | | | | (CVF618)] [Enterococcus |
| | | | | | | faecium Aus0085] |
| | | | | | | (tig/ropA) trigger factor [Trigger |
| | | | | | | factor (CVF149)] [Streptococcus |
| | | | | | | agalactiae 2603V/R] |
| | | | | | | (lisR) two-component response |
| | | | | | | regulator [LisR/LisK (CVF253)] |
| | | | | | | [Listeria innocua Clip11262] |
| | | | | | | (ACI49672) putative |
| | | | | | | housekeeping sortase [PilA-type |
| | | | | | | pili (PGS1, pilin gene clusters 1) |
| | | | | | | (AI132)] [Enterococcus faecium |
| | | | | | | str. E1165] |
| | | | | | | (pilA) PilA [PilA-type pili |
| | | | | | | (PGS1, pilin gene clusters 1) |
| | | | | | | (AI132)] [Enterococcus faecium |
| | | | | | | str. E1165] |
| | | | | | | (ACI49670) putative pilus- |
| | | | | | | dedicated sortase [PilA-type pili |
| | | | | | | (PGS1, pilin gene clusters 1) |
| | | | | | | (AI132)] [Enterococcus faecium |
| | | | | | | str. E1165] |
| | | | | | | (pilE) cell wall-associated |
| | | | | | | LPXTG-like protein [PilA-type |
| | | | | | | pili (PGS1, pilin gene clusters 1) |
| | | | | | | (AI132)] [Enterococcus faecium |
| | | | | | | str. E1165] |
| | | | | | | (ACI49669) hypothetical |
| | | | | | | hydrophobic peptide [PilA-type |
| | | | | | | pili (PGS1, pilin gene clusters 1) |
| | | | | | | (AI132)] [Enterococcus faecium |
| | | | | | | str. E1165] |
| | | | | | | (pilF) minor pilin subunit [PilA- |
| | | | | | | type pili (PGS1, pilin gene |
| | | | | | | clusters 1) (AI132)] |
| | | | | | | [Enterococcus faecium str. |
| | | | | | | E1165] |
| | | | | | | (tuf) elongation factor Tu [EF- |
| | | | | | | Tu (CVF587)] [Mycoplasma |
| | | | | | | gallisepticum str. R(low)] |
| | | | | | | (EFAU004_00491) periplasmic |
| | | | | | | solute binding protein [EfaA |
| | | | | | | (CVF610)] [Enterococcus |
| | | | | | | faecium Aus0004] |
| | | | | | | (rfbA-1) glucose-1-phosphate |
| | | | | | | thymidylyltransferase [Capsule |
| | | | | | | (CVF186)] [Streptococcus |
| | | | | | | gordonii str. Challis substr. |
| | | | | | | CH1] |
| | | | | | | (bsh) putative conjugated bile |
| | | | | | | acid hydrolase [Bile-salt |
| | | | | | | hydrolase (CVF250)] [Listeria |
| | | | | | | ivanovii subsp. ivanovii PAM |
| | | | | | | 55] |
| | | | | | | (ACI49664) putative pilus- |
| | | | | | | dedicated sortase [PilB-type pili |
| | | | | | | (PGS3) (AI133)] [Enterococcus |
| | | | | | | faecium str. E1165] |
| | | | | | | (pilB) PilB [PilB-type pili |
| | | | | | | (PGS3) (AI133)] [Enterococcus |
| | | | | | | faecium str. E1165] |
| | | | | | | (ACI49667) putative pilus tip |
| | | | | | | protein [PilB-type pili (PGS3) |
| | | | | | | (AI133)] [Enterococcus faecium |
| | | | | | | str. E1165] |
| | | | | | | (bsh) putative conjugated bile |
| | | | | | | acid hydrolase [Bile-salt |
| | | | | | | hydrolase (CVF250)] [Listeria |
| | | | | | | ivanovii subsp. ivanovii PAM |
| | | | | | | 55] |
| | | | | | | (sgrA) cell wall anchored |
| | | | | | | protein SgrA [SgrA (VF0540)] |
| | | | | | | [Enterococcus faecium DO] |
| LP28 | Ground | G+ | 99.9% | Lactobacillus | tetM [Clostridium | (SSU98_1513) phosphopyruvate |
| | Beef | rods | Lactobacillus | salivarius | difficile 630] | hydratase [Fibronectin-binding |
| | | | salivarius | | | protein (AI215)] [Streptococcus |
| | | | | | | suis 98HAH33] |
| | | | | | | (plr/gapA) glyceraldehyde-3- |
| | | | | | | phosphate dehydrogenase |
| | | | | | | [Streptococcal plasmin |
| | | | | | | receptor/GAPDH (CVF123)] |
| | | | | | | [Streptococcus agalactiae A909] |
| | | | | | | (groEL) chaperonin GroEL |
| | | | | | | [GroEL (CVF403)] [Clostridium |
| | | | | | | perfringens str. 13] |
| | | | | | | (clpC) endopeptidase Clp ATP- |
| | | | | | | binding chain C [ClpC |
| | | | | | | (VF0072)] [Listeria |
| | | | | | | monocytogenes EGD-e] |
| | | | | | | (hlyA) putative rRNA methylase |
| | | | | | | [Hemolysin (CVF589)] |
| | | | | | | [Mycoplasma mobile 163K] |
| | | | | | | (galU) UTP--glucose-1- |
| | | | | | | phosphate uridylyltransferase |
| | | | | | | [LOS (CVF494)] [Haemophilus |
| | | | | | | somnus 129PT] |
| | | | | | | (sspA) surface protein C |
| | | | | | | [Antigen I/II (AgI/II) family of |
| | | | | | | oral streptococcal adhesins |
| | | | | | | (CVF125)] [Streptococcus |
| | | | | | | sanguinis SK36] |
| | | | | | | (licD) lipopolysaccharide |
| | | | | | | choline phosphotransferase |
| | | | | | | [LOS (CVF494)] [Haemophilus |
| | | | | | | somnus 129PT] |
| | | | | | | (rffG) dTDP-glucose 46- |
| | | | | | | dehydratase [LOS (CVF494)] |
| | | | | | | [Haemophilus influenzae |
| | | | | | | PittGG] |
| | | | | | | (rmlA) putative glucose-1- |
| | | | | | | phosphate thymidyltransferase |
| | | | | | | [Capsule (CVF186)] |
| | | | | | | [Streptococcus mutans UA159] |
| | | | | | | (tuf) elongation factor Tu [EF- |
| | | | | | | Tu (CVF587)] [Mycoplasma |
| | | | | | | penetrans HF-2] |
| | | | | | | (tig/ropA) trigger factor [Trigger |
| | | | | | | factor (CVF149)] [Streptococcus |
| | | | | | | agalactiae A909] |

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A method for inhibiting the growth of food-borne pathogens, nosocomial pathogens and spoilage microorganisms, the method comprising the steps of:
contacting at least one lactic acid bacterium strain selected from the group consisting of Lactobacillus salivarius (L14, L28 and FS56), a mixture thereof; or a whey obtained from fermentation of the lactic acid bacterium strain with the microorganisms.

2. The method of claim 1, wherein the microorganisms are selected from the group consisting of gram-positive bacteria and gram-negative bacteria.

3. The method of claim 1, wherein the gram-positive bacteria are selected from the group consisting of Staphylococcus aureus, Listeria innocua, Listeria monocytogenes, Enterococcus faecium and Enterococcus faecalis.

4. The method of claim 1, wherein the gram-negative bacteria are selected from the group consisting of Escherichia coli and Salmonella Typhimurium.

5. The method of claim 4, wherein the Escherichia coli comprises the O157:H7 serotype.

6. The method of claim 1, wherein the microorganisms are selected from the group consisting of Aeromonas caviae; Aeromonas hydrophila; Aeromonas sobria; Bacillus cereus; Campylobacter jejuni; Citrobacter ssp.; Clostridium botulinum; Clostridium perfringens; Enterobacter ssp.; Enterococcus ssp.; Escherichia coli enteroinvasive strains; Escherichia coli enteropathogenic strains; Escherichia coli enterotoxigenic strains; Escherichia coli O157:H7; Klebsiella ssp.; Listeria monocytogenes; Plesiomonas shigelloides; Salmonella ssp.; Shigella ssp.: Staphylococcus aureus; Streptococcus ssp.; Vibrio cholerae; Yersinia enterocolitica. More preferably, the pathogenic-bacteria are Listeria monocytogenes, or strains MM13A, MM13B, P13, P17, Ecat, J19, J27, J35, J43, L14, L15, L17, L19, or LP28

7. The method of claim 1, wherein the at least one lactic acid bacterium strain is Lactobacillus salivarius (L28).

8. A method for reducing food-borne pathogens in a rendered food product comprising the steps of: combining a rendered food product and at least one lactic acid bacterium strain selected from the group consisting of Lactobacillus salivarius (L14, L28 and FS56), a mixture thereof; or a whey obtained from fermentation of the lactic acid bacterium strain with the microorganisms.

9. The method of claim 8, wherein the microorganisms are selected from the group consisting of Aeromonas caviae; Aeromonas hydrophila; Aeromonas sobria; Bacillus cereus; Campylobacter jejuni; Citrobacter ssp.; Clostridium botulinum; Clostridium perfringens; Enterobacter ssp.; Enterococcus ssp.; Escherichia coli enteroinvasive strains; Escherichia coli enteropathogenic strains; Escherichia coli enterotoxigenic strains; Escherichia coli O157:H7; Klebsiella ssp.; Listeria monocytogenes; Plesiomonas shigelloides; Salmonella ssp.; Shigella ssp.: Staphylococcus aureus; Streptococcus ssp.; Vibrio cholerae; Yersinia enterocolitica. More preferably, the pathogenic-bacteria are Listeria monocytogenes, or strains MM13A, MM13B, P13, P17, Ecat, J19, J27, J35, J43, L14, L15, L17, L19, or LP28.

10. The method of claim 8, wherein the rendered food product is a meat, fatty meat trimmings, sausages, toothpaste, a grease, or soap.

11. A method for increasing the storage time of a food by reducing the spoilage microorganisms in contact with the food comprising the steps of:
combining a food having one or more spoilage microorganisms with at least one lactic acid bacterium strain selected from the group consisting of Lactobacillus salivarius (L14, L28 and FS56), a mixture thereof; or a whey obtained from fermentation of the lactic acid bacterium strain with the one or more spoilage microorganisms to reduce the number of one or more spoilage microorganisms in contact with the food.

12. The method of claim 11, wherein the one or more spoilage microorganisms are selected from the group consisting of Aeromonas caviae; Aeromonas hydrophila; Aeromonas sobria; Bacillus cereus; Campylobacter jejuni; Citrobacter ssp.; Clostridium botulinum; Clostridium perfringens; Enterobacter ssp.; Enterococcus ssp.; Escherichia coli enteroinvasive strains; Escherichia coli enteropathogenic strains; Escherichia coli enterotoxigenic strains; Escherichia coli O157:H7; Klebsiella ssp.; Listeria monocytogenes; Plesiomonas shigelloides; Salmonella ssp.; Shigella ssp.: Staphylococcus aureus; Streptococcus ssp.; Vibrio cholerae; Yersinia enterocolitica. More preferably, the pathogenic-bacteria are Listeria monocytogenes, or strains MM13A, MM13B, P13, P17, Ecat, J19, J27, J35, J43, L14, L15, L17, L19, or LP28.

13. A method for reducing a pathogenic load in rendered food products comprising the steps of:
contacting at least one lactic acid bacterium strain selected from the group consisting of Lactobacillus salivarius (L14, L28 and FS56), a mixture thereof; or a whey obtained from fermentation of the lactic acid bacterium strain with the rendered food products having one or more pathogens to reduce the pathogenic load.

14. The method of claim 13, wherein the one or more pathogens are selected from the group consisting of Staphylococcus aureus, Listeria innocua, Listeria monocytogenes, Enterococcus faecium Enterococcus faecalis, Escherichia coli and Salmonella Typhimurium.

15. The method of claim 13, wherein the microorganisms are selected from the group consisting of Aeromonas caviae; Aeromonas hydrophila; Aeromonas sobria; Bacillus cereus; Campylobacter jejuni; Citrobacter ssp.; Clostridium botulinum; Clostridium perfringens; Enterobacter ssp.; Enterococcus ssp.; Escherichia coli enteroinvasive strains; Escherichia coli enteropathogenic strains; Escherichia coli enterotoxigenic strains; Escherichia coli O157:H7; Klebsiella ssp.; Listeria monocytogenes; Plesiomonas shigelloides; Salmonella ssp.; Shigella ssp.: Staphylococcus aureus; Streptococcus ssp.; Vibrio cholerae; Yersinia enterocolitica. More preferably, the pathogenic-bacteria are Listeria monocytogenes, or strains MM13A, MM13B, P13, P17, Ecat, J19, J27, J35, J43, L14, L15, L17, L19, or LP28.

16. The method of claim 13, wherein the Escherichia coli comprises the O157:H7 serotype.
